(19) United States
(12) Patent Application Publication
(10) Pub. No.: US 2015/0231301 A1
(43) Pub. Date: Aug. 20, 2015

(54) METHOD OF TREATMENT OF DISSECTED LYMPH NODE AND/OR VESSEL

(71) Applicant: CryoLife, Inc., Kennesaw, GA (US)

(72) Inventor: David Lang, Woodstock, GA (US)

(21) Appl. No.: 14/625,091

(22) Filed: Feb. 18, 2015

Related U.S. Application Data
(60) Provisional application No. 61/941,011, filed on Feb. 18, 2014.

(51) Int. Cl. A61L 26/00

(52) U.S. Cl. CPC A61L 26/0023; A61L 26/009; A61L 26/0085; A61L 2400/04

(57) ABSTRACT

Methods are provided that include applying to a surgical field in the patient following a lymphadenectomy or other procedure in which a lymph node or lymphatic vessel is dissected, a bioresorbable and biocompatible material in an amount effective to control oozing of lymphatic fluid. The material may be a hydrophilic polysaccharide material which, in contact with tissues in the surgical field, produces a gelled matrix that adheres to and forms a mechanical barrier with tissues in the surgical field to control oozing of lymphatic fluid. The hydrophilic polysaccharide material may include a modified starch such as a cross-linked carboxymethyl polysaccharide.

### CROSS-REFERENCE TO RELATED APPLICATIONS

This application claims priority to U.S. Provisional Patent Application No. 61/941,011, filed Feb. 18, 2014, which is incorporated herein by reference.

### TECHNICAL FIELD

The present invention is generally in the field of medical treatments and more particularly to methods of treatment of a patient following surgical dissection of lymphatic vessels or lymph nodes in the patient, e.g., a lymphadectomy.

### BACKGROUND

In open and laproscopic surgical procedures on patient, the surgeon may dissect one or more lymphatic vessels or lymph nodes. The dissection may be purposeful as in a lymphadectomy, or it may be inadvertent and unrecognized during the procedure. For example, lymphatic node dissection (LND) has been shown to improve staging of prostate cancer patients, and LND is recommended in intermediate and high risk prostate cancer patients undergoing radical prostatectomy. Lymphatic vessel or node dissections are particularly common with certain procedures where lymphatic tissues are involved. Examples of such procedures leading to lymphocysts are renal transplantation and radical pelvic surgery with lymph node removal because of prostatic or gynecologic cancer.

However, the lymphatic dissections may lead to increased drainage from site of incision, undesirably prolonging the patient's recovery and possibly leading to complications, such as lymphoceles. Radical retropubic prostatectomies in particular have pelvic lymphoceles rates that are undesirably high.

It therefore would be desirable to provide means to treat these lymphatic dissections, including but not limited to reducing the peri- and post-operative fluid ooze, reducing the duration of or need for drainage from the operative field, and ultimately reducing the frequency and/or severity of complications following open and laproscopic surgical procedures.

### BRIEF SUMMARY

Methods of treatment are provided for patients who have undergone a radical retropubic prostatectomy (RRP), a lymphadenectomy, or other procedure in which a lymph node or lymphatic vessel is dissected.

In one aspect, the method includes applying to a surgical field of the radical retropubic prostatectomy in the patient a hydrophilic polysaccharide material which, in contact with tissues in the surgical field, produces a gelled matrix that adheres to and forms a mechanical barrier with tissues in the surgical field to control oozing of lymphatic fluid. In one embodiment, the hydrophilic polysaccharide material comprises a cross-linked carboxymethyl polysaccharide. In a particular embodiment, the cross-linked carboxymethyl polysaccharide is a modified starch and is in the form of granules having an average length of from about 100 to about 500 micrometers.

In another aspect, the method includes applying to a surgical field in the patient following a lymphadenectomy or other procedure in which a lymph node or lymphatic vessel is dissected, a bioresorbable and biocompatible material in an amount effective to control oozing of lymphatic fluid. In one embodiment, the bioresorbable and biocompatible material comprises a hydrophilic polysaccharide, which may be a cross-linked carboxymethyl polysaccharide. In a particular embodiment, the cross-linked carboxymethyl polysaccharide is a modified starch and is in the form of granules having an average length of from about 100 to about 500 micrometers. In one embodiment, the bioresorbable and biocompatible material is applied to the surgical field after hemostasis is achieved. In one embodiment, the bioresorbable and biocompatible material is in a particulate form during the step of applying and subsequently becomes wetted and forms a gel. In one particular embodiment, the bioresorbable and biocompatible material is enzymatically degraded by alpha-amylase, glucoamylase, and macrophages within 2 to 4 days in vivo.

In still another aspect, a kit of parts is provided, which includes (a) a dispenser containing a bioresorbable and biocompatible material, and (b) instructions for using the dispenser to apply the bioresorbable and biocompatible material to a surgical field of and following a lymphadenectomy or other procedure in which a lymph node or lymphatic vessel is dissected.

### BRIEF DESCRIPTION OF DRAWINGS

FIG. 1 is a plan view of bellows type dispenser suitable for applying a hydrophilic polysaccharide material to a surgical field in a patient following a lymphadenectomy or other procedure in which a lymph node or lymphatic vessel is dissected.

FIG. 2 is a graph of showing the total fluid ooze per day (ml/24h) for RRP patients with and without treatment with a hydrophilic polysaccharide material.

FIG. 3 to FIG. 6 are graphs showing hemoglobin in the fluid drainage (mg/dl) for RRP patients with and without treatment with a hydrophilic polysaccharide material, for day 0, day 1, day 2, and day 3, respectively.

FIG. 7 to FIG. 10 are graphs showing the amount of fluid drainage (ml) per day for RRP patients with and without treatment with a hydrophilic polysaccharide material, for day 0, day 1, day 2, and day 3, respectively.

FIG. 11 is a graph showing the days of fluid drainage for RRP patients with and without treatment with a hydrophilic polysaccharide material.

FIG. 12 is a graph showing the surgeons' assessment of the field of surgery of the RRP patients after application of a hydrophilic polysaccharide material.

FIG. 13 is a graph showing the surgeons' assessment of his/her satisfaction with using the hydrophilic polysaccharide material in the RRP procedure on the patients.

### DETAILED DESCRIPTION

It has been discovered that applying a bioresorbable and biocompatible absorbent material to a surgical field in a patient following a lymphadenectomy or other procedure in which a lymph node or lymphatic vessel is dissected can be used to control oozing of lymphatic fluid. For example, use of the material has been observed to reduce the drainage volume the first day following a radical prostatectomy with pelvic lymph node dissection. In particular, the absorbent material contacts tissues in the surgical field and becomes wetted to produce a gelled matrix that adheres to and forms a mechanical barrier with tissues in the surgical field to control oozing of lymphatic fluid.

The term “patient” as used herein refers to a human or other mammal in need of treatment. In embodiments, the patient is a human in need of treatment to stop or stanch the flow or ooze of lymph and other fluids from a surgical site in the human patient following an open or minimally invasive procedure in which a lymph node or vessel has been dissected. In a particular embodiment, the patient is a human male who has undergone a radical retropubic prostatectomy.

As used herein, the phrases “applied to” and “applying to” convey that the one or more tissues are contacted with the bioresorbable and biocompatible absorbent material in any manner, including on the one or more tissues, in the one or more tissues, in a fluid or fluids associated with the one or more tissues, on a fluid or fluids associated with the one or more tissues, or a combination thereof. The act of “applying” may include spraying, pouring, sprinkling, or otherwise transferring the absorbent material from a container, or dispenser, to the selected tissues in the surgical field of the patient. A fluid is “associated” with one or more tissues when the fluid is in contact with the one or more tissues, has contacted the one or more tissues, is flowing (e.g., discharged, excreted, etc.) from the one or more tissues or a dissection therein, or a combination thereof.

The Absorbent Material, Kits, and Dispensing Systems for the Absorbent Material

The absorbent material used is the present methods preferably is a bioresorbable and biocompatible material. The bioresorbable absorbent materials are configured to degrade after a suitable period at the in vivo tissue site of application. In one embodiment, the absorbent materials forms a hydrogel that degrades over a desired time, e.g., several days or weeks, thereby eliminating the need to later remove the absorbent materials from the patient.

Generally, the absorbent materials provided herein may be in any physical form that permits application to one or more biological tissues. For example, the absorbent materials provided herein may be in the form of granules, particles, sheet, foam, gel, paste, mesh, film, colloid, bunting, pellets, or a combination thereof. The foam may be rigid or soft/flexible. The granules, in some embodiments, may be a powder. The particles, in other embodiments, may be a powder. In another embodiment, the material is provided in the form of a predefined structure, such as a film, sheet, ribbon, sponge, or other shaped article. Such an article may be flexible so as to easily conform to the tissue surfaces.

In one embodiment, the absorbent material is granulated. The granulation process produces granules, which are not substantially spherical. In embodiments, the granules are agglomerates. In some embodiments, the granules may have an average length of from about 1 to about 1000 micrometers. In further embodiments, the granules may have an average length of from about 100 to about 500 micrometers. In additional embodiments, the granules have an average length of from about 125 micrometers to about 250 micrometers. In still further embodiments, the granules have an average length of from about 10 micrometers to about 300 micrometers. As used herein, the phrase “average length” refers to the average size of the largest dimension of the granules.

In a preferred embodiment, the bioresorbable and biocompatible absorbent material comprises a hydrophilic polysaccharide material. In some embodiments, the absorbent material comprises a cross-linked carboxymethyl polysaccharide, such as a cross-linked carboxymethyl starch. The biocompatible cross-linked carboxymethyl starches may be plant-based. In one embodiment, potato starch is modified to produce the absorbent material. The cross-linked carboxymethyl polysaccharide may have a weight average molecular weight of from about 15,000 Da to about 3,000,000 Da.

The absorbent materials generally are sterile before use. For example, the absorbent materials may be sterilized before or after packaging. For example, the absorbent material may be provided in a dispenser or other storage container prior to use and then sterilized, such as by electron beam irradiation.

The absorbent materials may be provided with one or more biocompatible additives. In embodiments, the one or more additives include pharmaceutical agents. For example, the one or more additives may include one or more vasoconstrictors, clotting agents, antibiotics, antifungal agents, pain reducing agents, anti-inflammatory agents, tissue enzyme inhibitors, or a combination thereof. The one or more additives may be provided with the absorbent materials in any manner. For example, the one or more additives may be combined with the absorbent materials. In a particular example, both the additive and the absorbent material are in powder form, and the two powders are provided together as a powder blend. In another particular example, the one or more additives are provided in a container or on a bandage associated with the absorbent materials.

The absorbent materials described herein may be provided as part of a device, kit, or system for applying the absorbent materials.

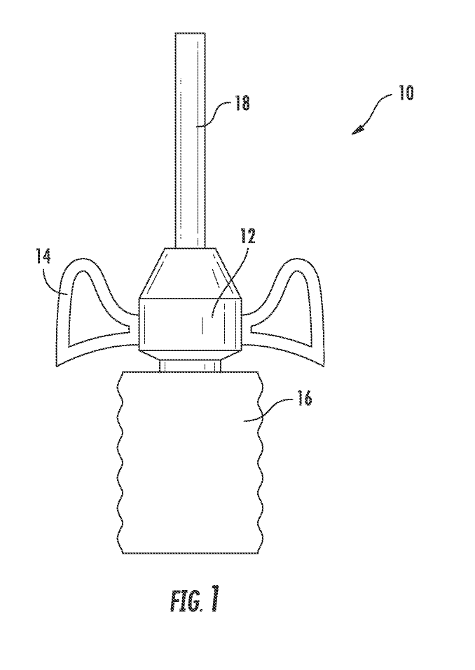

In a particular embodiment, the absorbent material is packaged, e.g., into a dispensing device, such as a bellows. In one embodiment, the absorbent materials of the device are provided in a particulate or granular form. The device may comprise a container of the absorbent materials. In one embodiment, the container is sized to contain from about 1 gram to about 15 grams of the granules, for example, from about 5 grams to about 10 grams of the granules. The container, in some embodiments, is a dispenser, such as a bellows pump. One example of a bellows pump is shown in FIG. 1. The bellows pump 10 has a compressible portion 16 in which a product is disposed, and the compressible (e.g., accordion) portion 16 may be compressed to push the product through the exit port 12 which is fitted with a dispenser tip 18 and loops 14 for the user's fingers. In other embodiments, the container includes the absorbent materials in free-flowing form, for example, wherein the container may include a spout or a series of holes to permit the pouring or sprinkling of the absorbent materials onto the target tissue site. The device, which may be in a kit form, also may include a tool that can be used to apply or spread the absorbent materials, such as an applicator.

Instructions for use (IFU) preferable are included in or with the packaging of the dispenser or kit. Such instructions describe how to apply the bioresorbable and biocompatible material to a surgical field of and following a lymphadenectomy or other procedure in which a lymph node or lymphatic vessel is dissected. The instructions may, for example, indicate that from about 5 grams to about 10 grams of the granules should be applied to the surgical field, e.g., to the complete surface of the fossa during/following an RRP. The material(s) of construction forming the packaging and the dispensing device desirably may be suitable for sterilization by irradiation. Polymeric, paper, and other suitable material of construction are known in the art.

In one embodiment, the bioresorbable and biocompatible material is a hemostat. For example, it may be a hemostatic powder as known in the art.

In one embodiment, the bioresorbable and biocompatible material comprises a polysaccharide. In a particular embodiment, the bioresorbable and biocompatible material is a starch, such as a starch or a modified starch, such as a carboxymethyl starch. In a preferred embodiment, the bioresorbable and biocompatible material is derived from one or more plants and not of animal origin, so as to reduce or eliminate the risk of allergic reactions. In one embodiment, the bioresorbable and biocompatible material consist essentially of a polysaccharide material derived from a plant.

In one embodiment, the bioresorbable and biocompatible particulate material comprises particles that are composed of a hydrophilic polysaccharide. The particles may be porous or non-porous. Such a material may form a gel upon absorption of aqueous fluids in the surgical site.

In a preferred embodiment, the bioresorbable and biocompatible particulate material is PerClot® polysaccharide particles (CryoLife Inc., Kennesaw, Ga., USA).

The Methods of Use and Treatment

Methods are provided for treating a patient who had a lymph node or lymph vessel dissected during a surgical procedure. The patient may be a human or other mammal. The methods include applying to a surgical field in the patient following a lymphadenectomy or other procedure in which a lymph node or lymphatic vessel is dissected, a bioresorbable and biocompatible material in an amount effective to control oozing of lymphatic fluid. The bioresorbable and biocompatible material may be applied to the field after hemostasis is achieved, for example using sutures, surgical adhesives, or other conventional means. In another embodiment, hemostasis is achieved at least in part concurrently with application of the bioresorbable and biocompatible material to the field.

The bioresorbable and biocompatible material may be applied into the surgical site by any suitable means, depending for example on the particular form of the material. For example, it may be manually placed (with or without the aid of an instrument or tool) onto the exposed tissue surfaces in the surgical field. If the material is in a particulate (e.g., granular) form, then it may be poured or sprinkled, or it may be applied using a bellows pump or other dispenser for delivering a dry powder. In other embodiments, the absorbent material is applied as a dry spray, moist spray, aerosol, or with aid of a carrier, such as a bandage, web, tape, fabric, foam, reticulated foam, film, or a combination thereof. For example, the absorbent material may be associated with a bandage by sprinkling the absorbent materials on the bandage, disposing the absorbent materials within the bandage, or a combination thereof. In other embodiments, the absorbent material is delivered with the aid of an endoscope, laparoscope, nasoscope, or celioscope.

The absorbent materials may form a hydrogel when contacting the lymph-containing fluid associated with the dissection of a lymph node or vessel. In embodiments, the hydrogel forms a cohesive layer. The cohesive layer, in some embodiments, is a matrix comprising lymph-containing fluid and the absorbent materials that may adhere to the one or more tissues. By adhering to the one or more tissues, the cohesive layer advantageously may reduce lymph fluid drainage and may prevent the ingress of infectious materials, such as bacteria, viruses, or a combination thereof.

In embodiments, the methods of use include applying the absorbent materials to one or more tissue sites in an amount effective for controlling lymph fluid. The phrase “controlling a fluid,” as used herein, includes absorbing the bodily fluid and/or reducing leakage of lymph, alone or in combination with blood or other bodily fluids. In one embodiment, the method includes applying from about 1 gram to about 15 grams of a modified starch, in a particulate form, to a surgical field where a lymph node or lymph vessel has been dissected, to control lymph fluid drainage. In a particular embodiment, the modified starch is a cross-linked carboxymethyl polysaccharide and from about 5 to 10 grams of granules comprising a cross-linked carboxymethyl polysaccharide are applied to control lymph fluid drainage. In one embodiment, the surgical field is in a patient who has undergone a radical retropubic prostatectomy (RRP).

In some embodiments, no pressure is applied to the absorbent materials after the absorbent materials are applied to one or more tissues. In other embodiments, pressure is applied to the absorbent materials after the absorbent materials are applied to one or more tissues. In these embodiments, pressure may be applied by any means, including a user's hand, a sheet material, a clamp or other tool, or a combination thereof. Non-limiting examples of sheet materials include a bandage, gauze, plastic film, applicator, or a combination thereof. In some embodiments, the methods provided herein include treating a patient of a surgical procedure involving dissection of a lymph node or vessel, such as a renal transplantation, radical retropubic prostatectomy, or radical pelvic surgery due to prostatic or gynecologic cancer. During a surgical procedure, the absorbent materials provided herein may be applied to one or more tissues affected by a lymphadenectomy or other procedure in which a lymph node or lymphatic vessel is dissected. In these embodiments, the absorbent materials provided herein are applied to the one or more tissues in an amount effective to control a lymphatic fluid, which may include controlling the excretion or oozing of the lymphatic fluid. In one embodiment, the absorbent materials provided herein may be applied to the one or more tissues after hemostasis is achieved, for example, by using sutures, surgical adhesives, or other conventional means. In another embodiment, the absorbent materials provided herein achieve hemostasis, at least in part, and control excretion or oozing of lymphatic fluid.

The methods described herein can be used with patients undergoing a variety of invasive procedures, such an open surgical procedure or a laproscopic surgical procedure, in which dissection of a lymph node or lymphatic vessel is known or likely to occur. In one embodiment, the patient has undergone a renal transplantation. In a particular embodiment, the patient has undergone radical pelvic surgery with lymph node removal because of prostatic or gynecologic cancer. In a particular embodiment, the patient has undergone a radical retropubic prostatectomy.

The present invention may be further understood with reference to the following non-limiting example.

### Example 1

### Treatment of Patients Undergoing Radical Retropubic Prostatectomy (RRP)

A prospective, randomized controlled clinical trial was conducted with 400 enrolled RRP patients. Increased postoperative drainage volume and pelvic lymphoceles occur rarely after RRP, but are associated with a prolonged hospital stay and increased rate of complications. The aim of the trial was to assess the impact of a polysaccharide hemostat, with primary endpoints of the study being postoperative fluid drainage composition, fluid drainage volume, and asymptomatic and symptomatic lymphocele formation rates.

A 1:1 randomization was used. Lymph node dissection was performed after removal of the prostate in a standardized fashion. Standard surgical techniques for hemostasis (i.e., sutures, coagulation, and titanium clipping) were used with half of the patients, and these standard techniques plus the additional application of 5 grams of PerClot® polysaccharide hemostat (particulate/granule form) applied in the field of the lymphadenectomy were used in the other half of the patients. The 5 grams of PerClot® polysaccharide granules were applied to the complete surface of the fossa prior to closure. Surgeons provided intraoperative rating of their satisfaction with fluid ooze control after intervention. In addition, total drainage volume was assessed after 3 hours and daily postoperatively. Pelvic drainage catheters were removed from the patients when the drainage volume dropped to less than 50 mL/24 hours. Drainage volume, percentage of hemoglobin, creatinine, triglycerides, hematoma and symptomatic and asymptomatic lymphoceles by ultrasound at day 3 and day 5 were recorded. Incidence symptomatic lymphoceles and classification were according to the Clavien-Dindo Classification System. Short term and long term complication rates were assessed after 1 week and again after 3 months (90 days).

Patient characteristics are shown in Table 1 below.

TABLE 1
RRP patient characteristics

| | With PerClot ® | Without PerClot ® | p-value |
|---|---|---|---|
| Age mean (SD) | 64.1 (7.4) | 65.4 (6.9) | 0.7 |
| BMI mean (SD) | 26.5 (3.2) | 26.7 (3.3) | 0.5 |
| Pre-OP PSA mean | 12.9 (14.9) | 12.5 (11.9) | 0.9 |
| (SD) | | | |
| Post-OP T-Stage (%) | | | 0.2 |
| ≦pT2c | 50.3 | 45.5 | |
| pT3a | 23.5 | 31.7 | |
| pT3b | 24.0 | 22.2 | |
| ≧pT4 | 2.2 | 0.6 | |
| Post-OP Gleason (%) | | | 0.4 |
| ≦sum 6 | 3.4 | 2.4 | |
| sum 7 | 81.1 | 86.2 | |
| ≧sum 8 | 15.6 | 11.4 | |
| LN yield (SD) | 20.3 (15.5) | 19.8 (11.0) | 0.5 |
| pN-Status (%) | | | 0.1 |
| pN0 | 73.2 | 80.8 | |
| pN1 | 26.8 | 19.2 | |
| pLND (%) | | | 0.6 |
| limited | 54.5 | 52.0 | |
| extended | 48.0 | 45.5 | |
| Aspirin use (%) | | | 0.3 |
| yes | 7.9 | 5.4 | |
| No | 92.1 | 94.6 | |
| Bloodloss mean (SD) | 874.6 (514.4) | 809.0 (438) | 0.4 |

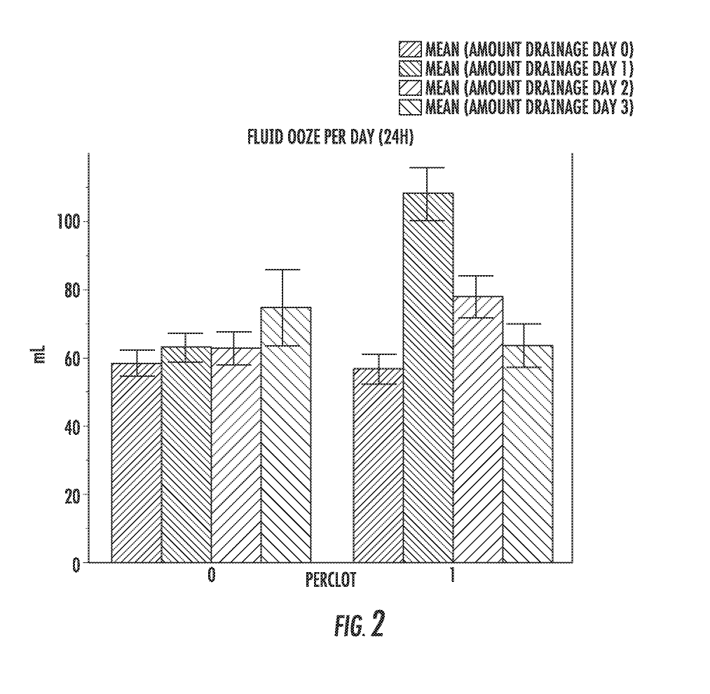

The results from the data collected from the patients are illustrated in FIGS. 2-13.

FIG. 2 shows volume of fluid ooze per day for each of the first four 24 hour periods following the RRP procedure. The bars on the left (“0” PerClot) are the mean values for the patients who were not treated with the PerClot® polysaccharide hemostat, and bars on the left (“1” PerClot) are the mean values for the patients who were treated with the PerClot® polysaccharide hemostat.

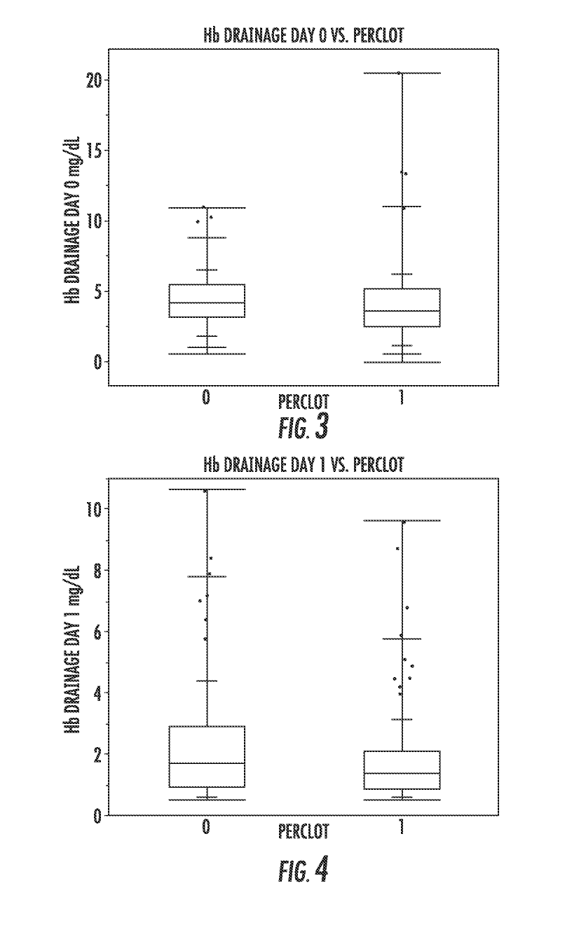

FIGS. 3-6 show the amounts of hemoglobin detected in the drainage fluid (mg/dl) for each of the first four 24 hour periods following the RRP procedure. The measured values are also given in Table 2 below.

TABLE 2
Hemoglobin in Drainage

| | | | | |
|---|---|---|---|---|
| | Number of | Median | | |
| PerClot ® | Patients | (mg/dl) | IQR | p value |

Day 0

| | | | | |
|---|---|---|---|---|
| 0 | 152 | 4.2 | 3.2-5.5 | |
| 1 | 157 | 3.6 | 2.4-5.1 | 0.007 |

Day 1

| | | | | |
|---|---|---|---|---|
| 0 | 158 | 1.7 | 1.0-2.9 | |
| 1 | 166 | 1.4 | 0.9-2.1 | 0.02 |

Day 2

| | | | | |
|---|---|---|---|---|
| 0 | 152 | 0.8 | 0.5-1.7 | |
| 1 | 162 | 0.6 | 0.5-1.6 | 0.2 |

Day 3

| | | | | |
|---|---|---|---|---|
| 0 | 60 | 0.5 | 0.5-0.7 | |
| 1 | 76 | 0.5 | 0.5-0.6 | 0.8 |

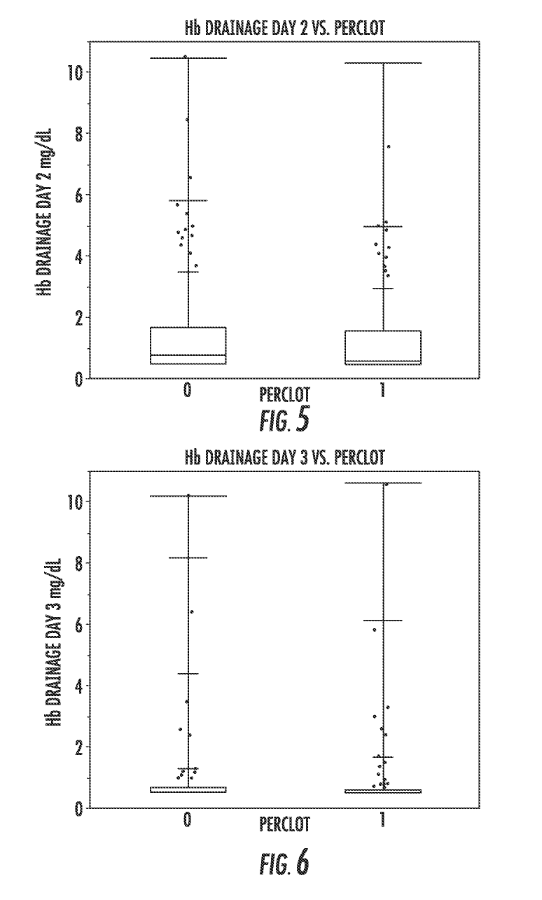

FIGS. 7-10 show the volume of fluid drainage collected for each of the first four 24 hour periods following the RRP procedure. The measured values are also given in Table 3 below.

TABLE 3
Drainage Volume

| | | | | |
|---|---|---|---|---|
| | Number of | Median | | |
| PerClot ® | Patients | (ml) | IQR | p value |

Day 0

| | | | | |
|---|---|---|---|---|
| 0 | 165 | 45 | 25-80 | |
| 1 | 169 | 35 | 18-75 | 0.043 |

Day 1

| | | | | |
|---|---|---|---|---|
| 0 | 165 | 50 | 30-75 | |
| 1 | 170 | 85 | 40-135 | <0.0001 |

Day 2

| | | | | |
|---|---|---|---|---|
| 0 | 164 | 45 | 25-89 | |
| 1 | 170 | 50 | 30-100 | 0.042 |

Day 3

| | | | | |
|---|---|---|---|---|
| 0 | 80 | 50 | 25-80 | |
| 1 | 94 | 50 | 29-80 | 0.7 |

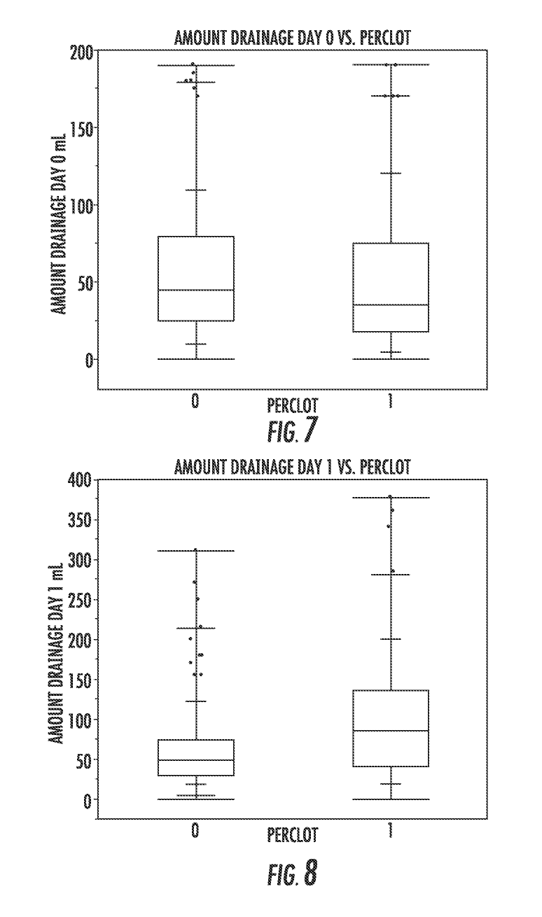

FIG. 11 shows the number of days of drainage until the drainage volume was low enough to remove the drainage catheter. The measured values are also given in Table 4 below.

TABLE 4
Days of Drainage

| | Number of | Median | | |
|---|---|---|---|---|
| PerClot ® | Patients | (days) | IQR | p value |
| 0 | 167 | 2 | 2-3 | |
| 1 | 178 | 3 | 2-3 | 0.2 |

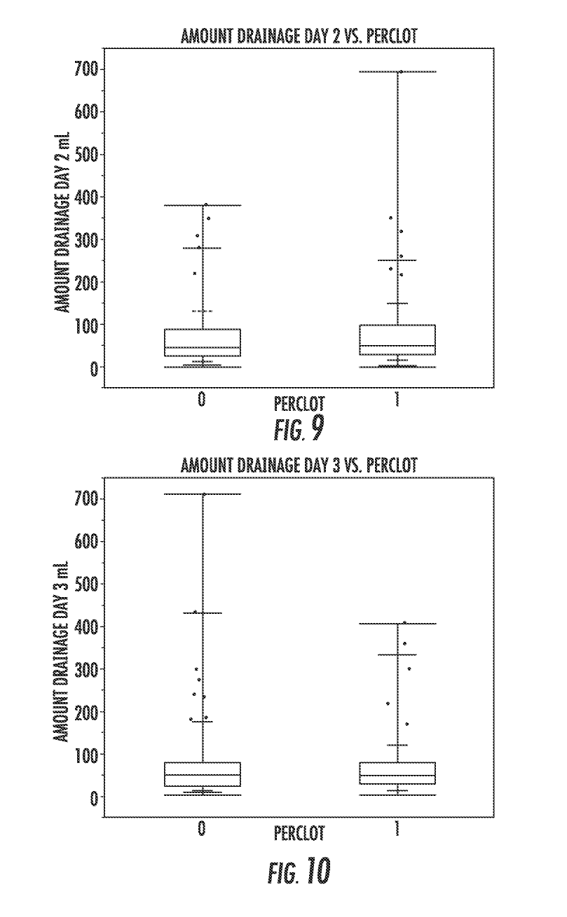

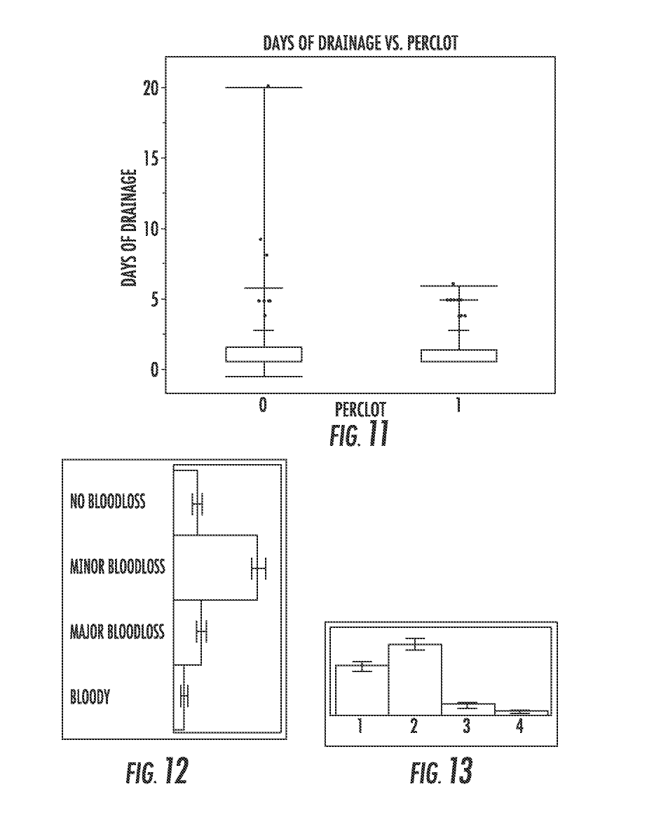

FIG. 12 shows the surgeons' assessment of the field of surgery after application of the PerClot® polysaccharide hemostat. FIG. 13 illustrates the surgeons' degree of satisfaction with the use/performance of the PerClot® polysaccharide hemostat, where “1” is “very satisfied”, “2” is “satisfied”, “3” is “not completely satisfied”, and “4” is “not satisfied”. Overall, 87% of the surgeons were satisfied or very satisfied with the effect of applying PerClot® polysaccharide hemostat to the surgical field, finding that it prevents diffuse bleedings, resulting in mostly no or minor blood loss seen.

Table 5 below shows the instances of lymphocele formation within 90 days of surgery. As the overall incidence rate of lymphoceles was low, no statistically significant reduction in the incidence of lymphoceles was achieved, despite the data demonstrating a lower number of lymphoceles in patients treated with 5 grams of PerClot® polysaccharide hemostat.

TABLE 5
Lymphocele Formation

| | | Lymphocele | Lymphocele | |
|---|---|---|---|---|
| Number of | | without | with surgical | |
| Patients | No lymphocele | intervention | intervention | Total |
| Without | 147 | 11 | 9 | 167 |
| PerClot ® | | | | |
| With PerClot ® | 167 | 6 | 6 | 179 |
| Total | 314 | 17 | 15 | 346 |

The data show a statistically significant reduction of drainage volume at day 0 can be achieved by using PerClot® polysaccharide hemostat during radical prostatectomy with pelvic node dissection. In addition, the use of PerClot® polysaccharide hemostat was found to help reduce hemoglobin count within the drainage fluid at day 0 and day 1. However, that particular effect was not effective enough to statistically significantly reduce the timespan in which drainage of the operation wound was necessary. This phenomenon may be attributable to the dosage of PerClot® polysaccharide hemostat used in the study, as indicated by a direct dosage dependency observed with patients not included in the study. A follow-up study is planned using 10 grams of PerClot® polysaccharide hemostat.

Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims.

I claim:

1. A method of treating a patient comprising:
performing a radical retropubic prostatectomy on the patient; and
applying to a surgical field of the radical retropubic prostatectomy a hydrophilic polysaccharide material which, in contact with tissues in the surgical field, produces a gelled matrix that adheres to and forms a mechanical barrier with tissues in the surgical field to control oozing of lymphatic fluid.

2. The method of claim 1, wherein the hydrophilic polysaccharide material comprises a cross-linked carboxymethyl polysaccharide.

3. The method of claim 2, wherein the cross-linked carboxymethyl polysaccharide is a modified starch and is in the form of granules having an average length of from about 100 to about 500 micrometers.

4. The method of claim 3, wherein from about 5 grams to about 10 grams of the granules is applied to the surgical field.

5. A method of treating a patient comprising:
applying to a surgical field in the patient following a lymphadenectomy or other procedure in which a lymph node or lymphatic vessel is dissected, a bioresorbable and biocompatible material in an amount effective to control oozing of lymphatic fluid.

6. The method of claim 5, wherein the bioresorbable and biocompatible material is in a particulate form.

7. The method of claim 5, wherein the bioresorbable and biocompatible material comprises a hemostat.

8. The method of claim 5, wherein the bioresorbable and biocompatible material comprises a hydrophilic polysaccharide.

9. The method of claim 8, wherein the hydrophilic polysaccharide material comprises a cross-linked carboxymethyl polysaccharide.

10. The method of claim 8, wherein the cross-linked carboxymethyl polysaccharide is a modified starch and is in the form of granules having an average length of from about 100 to about 500 micrometers.

11. The method of claim 5, wherein the bioresorbable and biocompatible material is PerClot® polysaccharides granules.

12. The method of claim 5, wherein the bioresorbable and biocompatible material comprises a microporous polysaccharide.

13. The method of claim 5, wherein the patient has undergone a radical retropubic prostatectomy.

14. The method of claim 5, wherein the bioresorbable and biocompatible material is applied to the surgical field after hemostasis is achieved.

15. The method of claim 5, wherein the bioresorbable and biocompatible material is in a particulate form during the step of applying.

16. The method of claim 15, wherein following the step of applying the particulate form becomes wetted and forms a gel.

17. A method of treating a patient comprising:
performing a lymphadenectomy on the patient; and then
applying to a surgical field of the lymphadenectomy a bioresorbable and biocompatible material in an amount effective to control oozing of lymphatic fluid.

18. The method of claim 17, wherein the bioresorbable and biocompatible material comprises a hydrophilic polysaccharide containing no animal or human components, wherein upon contact with aqueous fluid in the surgical field, the hydrophilic polysaccharide produces a gelled matrix that adheres to and forms a mechanical barrier with tissues in the surgical field.

19. The method of claim 18, wherein the applied the bioresorbable and biocompatible material is enzymatically degraded by alpha-amylase, glucoamylase, and macrophages within 2 to 4 days in vivo.

20. The method of claim 18, wherein from about 5 grams to about 10 grams of the hydrophilic polysaccharide is applied to the surgical field.

21. A kit of parts comprising:
a) a dispenser containing a bioresorbable and biocompatible material, and
b) instructions for using the dispenser to apply the bioresorbable and biocompatible material to a surgical field of and following a lymphadenectomy or other procedure in which a lymph node or lymphatic vessel is dissected.

22. The kit of claim 21, wherein the bioresorbable and biocompatible material is in particulate form.

23. The kit of claim 22, wherein the dispenser comprises a bellows and a nozzle.

24. The kit of claim 23, wherein the bioresorbable and biocompatible material is PerClot® polysaccharides granules, and the dispenser contains from about 5 grams to about 10 grams of the polysaccharides granules.
